(19) United States
(12) Patent Application Publication
(10) Pub. No.: US 2002/0160064 A1
(43) Pub. Date: Oct. 31, 2002

(54) Cosmetics containing isoflavone aglycones

(76) Inventors: Fred Zulli, Kuttigen (CH); Daniel Schmid, Brugg (CH); Reto Muggli, Olten (CH); Christiane Hanay, Aarau (CH)

Correspondence Address: Paul J. Maginot, Maginot, Moore & Bowman, Bank One Center/Tower, 111 Monument Circle, Suite 3000, Indianapolis, IN, 46204-5115, US

(21) Appl. No.: 10/080,942

(22) Filed: Feb. 22, 2002

(30) Foreign Application Priority Data
Feb. 26, 2001 (CH) 0349/01

(51) Int. Cl. A61K 35/78; A61K 6/00

(52) U.S. Cl. 424/757; 424/725; 424/401

(57) ABSTRACT

Cosmetics comprising at least one isoflavone aglycone, particularly genistein and/or daidzein, in a biologically active form as active component which is incorporated into liposomes are effective in the treatment of cellulitis, in the treatment of female or male skin after the climacteric, particularly of female skin after the menopause, for increasing the size and firmness of female breasts, and for whitening the skin. Preferably, said isoflavone aglycones incorporated into liposomes are combined with algal extract.

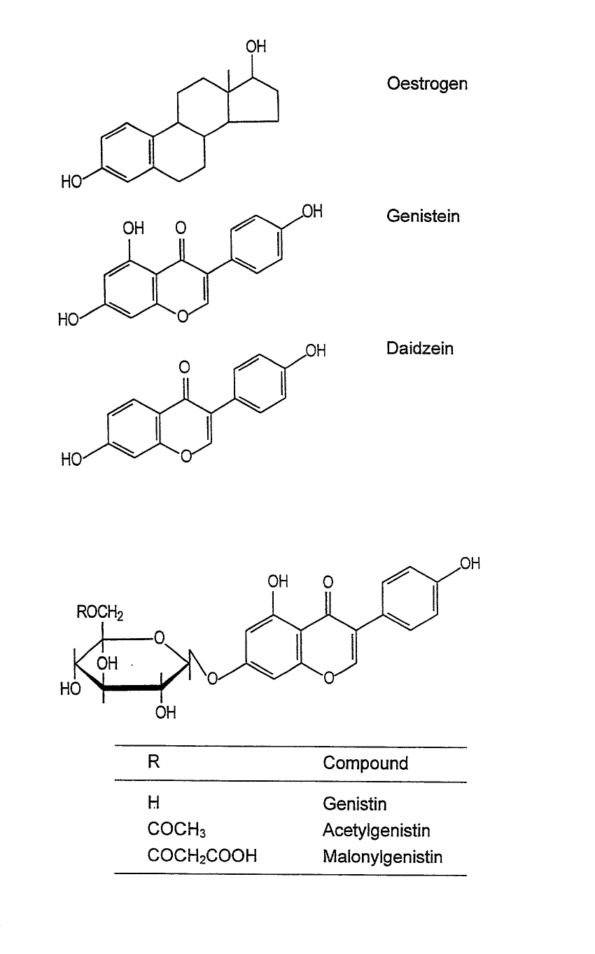

### FIELD OF THE INVENTION

The present invention is concerned with cosmetics containing at least one isoflavone aglycone in a biological active form as active component.

### BACKGROUND OF THE INVENTION

As is well known, human skin consists of two layers, i.e. of the outer thinner epidermis and the underlying thicker dermis. The epidermis comprises as main cell type keratinocytes which are cornified in the outermost zone, thus protecting the skin against drying and mechanical and chemical influences. The dermis is rich in structure components, such as collagen and elastin proteins as well as proteoglycans und sugar/protein complexes. These structure components are formed by fibroblast cells and confer to the skin the necessary thickness as well as elasticity.

The phenomenon of skin aging manifests by the formation of wrinkles and a general weakening of the skin, as well as by a reduction of its elasticity and firmness. On one hand, skin aging is a time depending process which however may be accelerated by external factors such as ultraviolet radiation. Ultraviolet radiation produces reactive oxidizing products starting mechanisms which also are active in the time depending skin aging. These mechanisms taking place in the dermis result in a reduced creation of new structure elements and in an accelerated degradation of existing structure elements.

Women, after the menopause, are subject to a sudden skin aging. The phenomenon of the menopause is provoked by a drastic reduction of the production of the female sex hormone oestrogen. Thus, this fact is called “hormone controlled skin aging”.

Antioxidants are used as active components against ultraviolet radiation induced skin aging. Usual active components against skin aging in general are α-hydroxy acids and retinic acid. However, these compounds produce side effects such as skin tension and skin irritations. Another attempt in the therapy of skin aging is to use substances which influence the regulation of the synthesis of dermis structure elements. Desirable are active compounds which promote the synthesis of such structure proteins and/or reduce their degradation. Oestrogens and oestrogen like compounds are known for this purpose.

Various studies have shown that the thickness and the elasticity of the skin of women after the menopause can be maintained by a therapy with oral oestrogen (Hormone Substitute Therapy). Therapies with externally applied oestrogen also proved successful for combating the sudden skin aging after the menopause. However, the constant intake of oestrogen produces an increased risk of cancer of breast and of carcinoma of the uterus. Recently, synthetic oestrogen-like compounds (e.g. Raloxifen®) were introduced which are said to produce no cancer risk.

At present, isoflavones are intensively tested as a natural alternative of the hormone substitute therapy. Isoflavones are a class of plant oestrogens. They are very similar to human oestrogen with respect to structure and function. However, in contrast to the latter, they do not produce any cancer risk but even have an anti-cancerogenous effect, so that they are sold in functional food products.

Soybeans are very rich in isoflavones. They contain the isoflavone aglycones genistein, daidzein and glycitein as well as the corresponding β-glycosides genistin, daidzin and glycitin. Non-fermented soya products mainly contain the polar and thus water soluble glycosides. If soya is fermented, the sugars are cleaved by bacterial enzymes. Only aglycones have an oestrogen-like activity. Studies on cell cultures show that isoflavones stimulate the synthesis of collagen [Kawashima et al., Biochemical Pharmacology, Vol. 51 (1996), Page 133] and reduce the production of collagen decomposing enzymes (matrix metalloproteinases) [Shao et al., Anticancer Research, Vol. 18 (1998), Page 1435]. Thus, genistein replaces, with respect to the skin structure, the functions of the oestrogen missing after the menopause. Isoflavones also have a positive effect on the skin of males. By promoting the synthesis of skin structures, they combat the age depending skin thinning which also occurs in males. The female sexual hormone oestrogen cannot be applied to males. Depending on the tissue, the effect of isoflavones is either oestrogenic (bones, blood circulation, brain) or anti-oestrogenic (breast, uterus) [Maroulis, Annals of the New York Academy of Sciences, Vol. 900 (2000), Page 413)]. Thus, they do not show undesired hormonal activity on males.

Patents and patent applications describing the use of isoflavones in cosmetics have been published. JP-58225004 and JP-7157494 describe isoflavones as skin whitening agent. DE-4432947 describes isoflavones for treating the skin against teleangiectatic rosacea, starburst varices, melanomas, alopecia, acne, fatty skin and pigment spots, as well as hair restorer and radical inhibitor. U.S. Pat. No. 5,824,702 describes genistein as preventive remedy against ultraviolet radiation induced skin damages. WO-99/04747 describes the isoflavone resveratrol as remedy in the therapy of aged and light-damaged skin. WO-99/36050 shows the effect of isoflavones against ultraviolet radiation induced suppression of the immune system as well as against ultraviolet radiation induced skin damage in general. FR-2782919 maintains that a mixture of retinic acid with isoflavones can delay the appearing of signs of skin aging. It is explained that this mixture shows a synergetic effect inasmuch as the degradation of dermis structure proteins is reduced. U.S. Pat. No. 6,060,070 describes the use of isoflavones against skin aging in females and males based on the oestrogen-like activity of the isoflavones.

The above mentioned publications make no difference between the use of isoflavones in the form of sugar derivatives and tat of aglycones. However, in topic applications this is a crucial point for the following reasons. Isoflavones as sugar derivatives do not have any physiological effect. In nature, the isoflavones are present in the plant as sugar derivative. For activation, the sugars are compulsorily to be cleaved. If isoflavones are applied orally, e.g. in the form of a supplemented foodstuff, they need not to be activated, since in the intestine the sugars are cleaved by hydrolytic enzymes of the intestinal cells and of the intestinal flora. However, if isoflavones are applied in cosmetics they are compulsorily to be activated first, i.e. they are to be brought into the aglycone form, since there are no hydrolytic enzymes on the skin. In the form of sugar derivatives the isoflavone would not penetrate into the deeper skin layers, i.e. the dermis, since the fat layer formed by the epidermis lets pass only the apolar water-insoluble aglycones. Thus, said completely water-insoluble aglycone can only by introduced into cosmetics in combination with a solubilizer.

### OBJECTS OF THE INVENTION

A first object of the present invention is to eliminate said disadvantages of the prior art.

Another object of the present invention is to provide highly effective cosmetics useful in the treatment of signs of skin aging in general, and in particular of the sudden skin aging of women after the menopause, in the treatment of cellulites and acne, for increasing the size and firmness of female breasts, and the whitening of the skin.

The forgoing and further objects, advantages and features will be apparent form the following specification.

### SUMMARY OF THE INVENTION

To meet these and other objects, the invention provides a cosmetic comprising at least one isoflavone aglycone in a biologically active form as active component, said at least one isoflavone aglycone being incorporated into liposomes.

Thus, the invention is based on a novel formulation combining isoflavone aglycones with a carrier system. The latter comprises the phospholipid lecithin which in aqueous solution forms liposomes. The water-insoluble aglycones settle in the lecithin double-lipid membrane of the liposomes. Incorporated in this way into the liposomes, the aglycones can stably be introduced into aqueous cosmetics. When these products are applied to the skin, the aqueous component gradually evaporates, the liposomes disintegrate, the lecithin fuse together with the fatty layer of the skin, and the liberated aglycones can penetrate through said fatty layer into the deeper skin layers.

The isoflavones which are useful in the present invention belong to the group consisting of genistein, daidzein, glycitein, formononetin, tectorigenin, irigenin, biochanin A, O-desmethylangolensin, equol, orobol, santal, pratensin and apiosylpuerarin. The preferred compounds are genistein and daidazein.

Preferably, the cosmetics further comprise at least one algal extract, particularly an extract of algae of the genus Spirulina.

Preferably, the concentration of said isoflavone aglycones is from 1 to 500 mg per kg of the cosmetic, particularly from 20 to 100 mg per kg of the cosmetic.

The following specification also describes the preparation of a aglycone active component from the isoflavones genistin, daidzin and glycitin.

The emergence of cellulitis is mainly depending on the sex-determined anatomic structure of the skin and the influence of sexual hormones. In males, in the upper subcutic layers connective tissue septua overcross themselves scissor like thus clip like including the fat cells. If then the skin is pressed, the fat chambers are retained by the connective tissue grid. The epidermis corium layer is thicker and the subcutic layer is thinner than in females. The outer subcutic layer consists of vertical fat cell chambers which are tubularly separated from each others by radially extending convective tissue septa. The outer boundary of these fat tubes is the thinner and weaker epidermal corium layer which cushion-likely vaults when the skin is squashed. Approximately form the 30th year of one's life on, the epidermal corium layer becomes thinner, the elastic and collagenous fibbers become weaker and less numerous, whereas the subcutis becomes thicker, which favors a cushion-like skin relief.

Algal extracts become continually more important as alternative raw materials in cosmetics. In particular, the blue-green microalga of the genus Spirulina is known for its high content of polyunsaturated fatty acids, essential amino acids, minerals and natural antioxidants, such as α-tocopherol and β-carotene. Algal extracts are used as radical inhibitors in anti-aging cosmetics. The effectiveness of algal extracts in the treatment of cellulitis is due to its lipolytic activity and to an improved transportation of waste products.

The use of isoflavones in the cellulitis therapy is a totally new attempt. It is known that a hormone substitute therapy with oestrogen produces an improved skin structure. However, a topical treatment of cellulitis with oestrogen is questionable since cellulitis is originally induced just by oestrogen. Moreover, it is known that the oestrogen-like isoflavones have anti-oestrogenic activity in breast and uterus. This fact, combined with the fact that genistein stimulates the synthesis of collagen and reduces the production of collagen-degrading enzymes (matrix metalloproteinases), provide isoflavones a high potential in the treatment of cellulitis. A topical treatment with isoflavones should lead to a thickening and strengthening of the dermis layer through which the cushion-like vaulting of the fat cell chambers can be reduced.

Genistein causes in the human body a reduction of the fatty tissue. This oestrogen-independent effect is an additional important mechanism in the treatment of cellulitis by genistein. Said decomposition of fat is due to the fact that genistein inhibits the enzyme phosphodiesterase [Kuppusamy and Das, Biochemistry and Pharmacology, Vol. 44, Page 1307]. This inhibition makes that more cyclic adenosinmonophosphate, i.e. a messenger compound within the cell stimulating the enzyme lipase to decompose fat, is present in the cell. A general decomposition of fat is also caused by the fact that genistein inhibits the propagation of precursor fat cells [Harmon und Harp, American Journal of Physiological Cell Physiology, Vol. 280, Page C807].

There is an extensive literature on the effect of isoflavones in inhibiting the cancer of breast. However, isoflavone can influence the metabolism of healthy tissue. At present, several plant extract products containing plant oestrogens, such as e.g. form Black Cohosh or from Pueraria mirifica, are offered for increasing the size and firmness of female breasts. The effect of such products is said to be due to a stimulation for the formation of the breast tissue and an enlargement of the milk duct. A scientific publication [McMichael-Phillips et al., American Journal of Clinical Nutrition, Vol. 68 Suppl. (1998), Page 1431] has shown that increased genistein and daidazein blood values occurring after soya complement nutrition significantly stimulated the growth of breast tissue.

The finding that the isoflavones stimulate the growth of healthy breast tissue, in combination with the fact that they thicken and strengthen the dermis layer of the breast skin, indicate the use of isoflavones for increasing the size and firmness of female breasts.

Acne vulgaris, the most frequently occurring skin disease, is generated by the hormonal changeover during puberty and some times during menstruation or pregnancy. The basic problem in its pathogenesis is an excessive production of sebum. Sebaceous glands are activated by androgens, the so-called “male” hormones. Thus, an increased androgen level is often the cause of acne. Acne can effectively be combated by oestrogenic substances which normalize the androgen level. However, hormones are not allowed in cosmetics, and oestrogens raise the risk of cancer. Isoflavones are natural oestrogen-like substances which also should normalize the androgen level.

Isoflavones produce, beside the fat degrading effect, further non-oestrogenous effects. The latters are due to the property of the isoflavones to inhibit certain enzymes, i.e. the so-called protein kinases. The enzyme protein kinase plays a deciding role in the synthesis of color pigments in melanoma cells. The protein kinase activates the enzyme tyrosinase which has a key position in the synthesis if color pigments. In most cases, the substances used so far as skin whiteners act by inhibitation of the enzyme tyrosinase. Thus, in the field of skin whitening isoflavones represent a new group of active agents.

Active soya isoflavone components are available on the market. However, they contain the plant oestrogens in the biologically inactive form of glycosides. This is of no importance if the active compounds are used as nutritional complement in functional food product, since the enzymes are converted by the intestinal flora into the active aglycones. However, if these isoflavone compounds are applied to the skin, they remain inactive.

The present invention now provides for the first time an active isoflavone composition which contains plant oestrogens in the active aglycone form, said water-insoluble aglycones being incorporated into a liposomal structure, thus providing a galenic which allows the active component to be incorporated in cosmetic formulations. At the same time, said liposomal structure confers an excellent penetrative quality into the skin cells.

Said active liposomal aglycone compound can be prepared by treating soya isoflavone material with a β-glucosidase enzyme for 24 to 500 hours at 20 to 60° C. Thereafter, the obtained water-insoluble aglycones are separated by centrifugation or filtration. Then, the aglycones can be dissolved in absolute ethanol. For introducing the aglycones into liposomes, said ethanolic solution is homogenized together with an aqueous lecithin dispersion.

Alternatively, the active liposomal aglycone compound can be produced by using a fermented soya fraction as starting material. By the fermentation, the isoflavone glycosides are converted into the corresponding aglycones. Thus, e.g. the press cake as it is obtained after a Moromi fermentation in the manufacture of soya sauces is a suitable starting material. The aglycones can be extracted with absolute isopropanol. For introducing the aglycones into liposomes, said isopropanolic solution is homogenized or stirred together with an aqueous lecithin dispersion.

Furthermore, the liposomal aglycone compound can be produced from soya molasses as starting material. Soya molasses is a by-product from the production of soya protein concentrate. In the production of said protein concentrate, soya flour is first extracted with an aqueous alcoholic solution for removing bitter principles and undesired odors having the typical beany note. Upon recycling of the alcohol, the soya molasses yields a concentrate of sugars, bitter principles and isoflavones. For the preparation of the liposomal aglycone active compounds, it can be proceeded as described above with respect to the soya isoflavone material.

The formulas of some compounds useful in the present invention are represented in the accompanying drawing.

The following examples and formulations will explain the present invention more in detail.

Unless otherwise stated, all numerals given below are percents by weight. The indication of the ingredients is mainly made according to the INCI (International Cosmetics Ingredients) nomenclature.

### EXAMPLES

### EXAMPLE 1

### Preparation of a Soya Isoflavone Aglycone Active Compound in Ethanol

(a) From a soya fraction enriched in isoflavones

A soya fraction enriched in isoflavones was used as starting material. For hydrolysis, 50 g of this material was introduced into 1 liter of potassium sorbate (0,6 per cent, pH 5,0) and treated with 300 mg β-glucosidase at 37° C. for 4 days. The precipitated water-insoluble aglycones were separated by filtration and thereafter rinsed twice with water. For extracting of the aglycones, the filtrate was dissolved in 420 ml of ethanol and stirred for 2 hours at 25° C. The remaining ethanol-insoluble material was separated by filtration. Analysis of the ethanolic extract by High Performance Liquid Chromatography [HPLC] (C18 column) showed that 86 percent of the original genistine glycoside and 50 percent of the original daidzin glycoside could be recovered as their aglycones.

(b) From a press cake obtained from the soya sauce production after a Koji and Moromi fermentation

Analysis of the press cake by High Performance Liquid Chromatography [HPLC] (C18 column) showed that it contained 0,07 percent of genistein and 0.04 percent of daidzein. Prior to the isolation of the isoflavone aglycones, the press cake was washed with water. For this, 400 g of press cake were mixed with 4 liters of water and stirred for 2 hours at 25° C. The water-insoluble material was separated by filtration, absorbed in 2 liters of ethanol, and stirred for 2 hours at 25° C. The ethanolic extract was separated from the solids by filtration. This extraction process was repeated once. The ethanolic extracts were combined and concentrated in a Rotavapor® vacuum evaporator to one hundredth.

(c) From soya molasses, a by-product form the production of soya protein concentrates

For the hydrolysis, 100 g of soya molasses were absorbed in 1 liter of potassium sorbate (0.6 percent, pH 5.0) and treated with 300 mg β-glucosidase at 37° C. for 4 days. Thereafter, the process was continued as described sub (a).

### EXAMPLE 2

### Preparation of an Soya Isoflavone Aglycone/Liposomes Active Compound

For the preparation of the aglycone/liposomes solution, 10 ml of soya isoflavone aglycone active compound were prepared according to one of the methods described sub (a) to (c) of Example 1, then mixed with 10 ml of 50 percent lecithin solution in ethanol, mixed and stirred in 80 ml of water, and five times homogenized at 1200 bar (120 Pa). The particle diameter of the liposomes was 120±20 nm.

EXAMPLE 3
Preparation of a soya aglycone/algal extract active compound combination

| | |
|---|---|
| Aqueous algal extract from Spirulina platensis | 40.0% |
| Soya isoflavone aglycone/liposomes active compound in | 20.0% |
| ethanol (2% aglycones) | |
| Polysorbate 80 | 20.0% |
| Preservatives, Aqua | ad 100.0% |

FORMULATIONS
1. Anti-cellulitis gel

| | |
|---|---|
| Glucose | 4.0% |
| Aluminium Starch Octenyl Succinate | 1.5% |
| Soya isoflavone aglycone/algal extract active compound | 5.0% |
| combination (0.25% aglycones) | |
| Polysorbate 20 | 0.6% |
| Carbomer | 0.5% |
| Ginkgo biloba extract | 0.5% |
| Preservatives, Sodium Hydroxyde Solution, Perfume, Aqua | ad 100.0% |

2. Anti-cellulitis cream

| | |
|---|---|
| Caprylic/Capric Triglyceride | 12.0% |
| Hydrogenated Coco-Glyceride | 3.0% |
| Polyglyceryl-3-Methylglucose Distearate | 3.0% |
| Soya isoflavone aglycone/algal extract active compound | 5.0% |
| combination (0.4% aglycones) | |
| Glyceryl Stearate | 6.0% |
| Cetyl Alcohol | 1.0% |
| Glyceryl Polymethacrylate | 1.0% |
| Stearyl Alcohol | 1.0% |
| Ginkgo biloba Extract | 0.5% |
| Preservatives, Perfume, Aqua | ad 100.0% |

3. Anti-cellulitis intensive concentrate

| | |
|---|---|
| Soya isoflavone aglycone/algal extract active compound | 3.0% |
| combination | |
| Pentylene Glycol | 2.0% |
| Carnitine | 0.2% |
| Caffeine | 0.1% |
| Ruscus aculeatus Extract | 0.1% |
| Butylene Glycol | 2.0% |
| Glycerin | 2.0% |
| Polysorbate-20 | 1.0% |
| Xanthan Gum | 0.3% |
| Preservatives, Perfume, Aqua | ad 100.0% |

4. Anti-cellulitis 2-phase bath

| | |
|---|---|
| Paraffinum Liquidum | 20.0% |
| Sodium Laureth Sulfate | 8.4% |
| Propylene Glycol | 8.0% |
| Cocamidopropyl Betaine | 3.0% |
| Sodium Chloride | 2.5% |
| Glycerin | 2.0% |
| Isohexadecane | 1.0% |
| Soya isoflavone aglycone/algal extract active compound | 3.0% |
| combination | |
| Carnitine | 0.2% |
| Caffeine | 0.1% |
| Ruscus aculeatu Extract | 0.1% |
| Preservatives, Perfume, Aqua | ad 100.0% |

5. Anti-cellulitis hydro intensive massage cream

| | |
|---|---|
| Isononyl Isononanoate | 4.0% |
| Glycerin | 4.0% |
| Paraffinum Liquidum | 4.0% |
| Arachidyl Glycoside, Arachidyl Alcohol | 5.0% |
| Soya isoflavone aglycone/algal extract active compound | 3.0% |
| combination | |
| Carnitine | 0.2% |
| Caffeine | 0.1% |
| Ruscus aculeatus Extract | 0.1% |
| Squalane | 2.0% |
| Myristyl Glycoside, Myristyl Alcohol | 2.0% |
| Cyclomethicone | 2.0% |
| Butylene Glycol | 2.0% |
| Carbomer | 0.3% |
| Preservatives, Perfume, Aqua | ad 100.0% |

6. Facial cream against signs of skin aging for women after the menopause

| | |
|---|---|
| Octyldodecanol | 5.0% |
| Paraffinum Liquidum | 3.0% |
| Isopropyl Isostearate | 3.0% |
| Cetyl Alcohol | 2.5% |
| Stearyl Alcohol | 2.0% |
| Dicaprylyl Ether | 2.0% |
| Palmitic/Stearic Acid | 2.0% |
| Polyglyceryl-3-Methylglucose Distearate | 2.0% |
| Propylene Glycol | 2.0% |
| Soya isoflavone aglycone/liposomes active compound | 5.0% |
| (0.2% aglycones) | |
| Glycerin | 1.0% |
| Xanthan Gum | 0.2% |
| Preservatives, Perfume, Aqua | ad 100.0% |

7. Body cream against signs of skin aging for women after the menopause

| | |
|---|---|
| Cetearyl Glycoside | 5.0% |
| Diispropyl Dimer Dilinoleate | 5.0% |
| Paraffinum Liquidum | 5.0% |
| Glycerin | 2.0% |
| Butyrospermum Parkii | 2.0% |
| Soya isoflavone aglycone/liposomes active compound | 0.1% |
| (0.2% aglycones) | |
| Dimethicone | 1.0% |
| Squalane | 0.5% |
| Carbomer | 0.2% |
| Preservatives, Perfume, Aqua | ad 100.0% |

8. Serum against signs of skin aging for women after the menopause

| | |
|---|---|
| Dicaprylyl Ether | 5.0% |
| Glycerin | 3.0% |
| Distarch Phosphate | 2.5% |
| Trilaureth-4 phosphate | 2.5% |
| Dimethicone | 3.0% |
| Butyl Alcohol | 1.0% |
| Soya isoflavone aglycone/liposomes active compound | 20.0% |
| (0.2% aglycones) | |
| Carbomer | 0.5% |
| Preservatives, Sodium Hydroxyde Solution, Perfume, Aqua | ad 100.0% |

9. Breast cream for increasing the size and firmness of female breasts

| | |
|---|---|
| Dicaprylyl Ether | 4.0% |
| Isononyl Isononanoate | 3.0% |
| Arachidyl Glycoside | 3.0% |
| Octyldodecanol | 3.0% |
| Myristyl Glycoside | 2.0% |
| Soya isoflavone aglycone/liposomes active compound | 5.0% |
| (0.2% aglycones) | |
| Dimethicone | 1.0% |
| Carbomer | 0.5% |
| Preservatives, Sodium Hydroxyde Solution, Perfume, Aqua | ad 100.0% |

10. Anti-acne facial emulsion

| | |
|---|---|
| Polyacrylamide | 3.5% |
| Hydrogenated Polyisobutene | 3.5% |
| Glycerin | 2.0% |
| Propylene Glycol Dicaprylate/Dicaprate | 2.0% |
| Soya isoflavone aglycone/liposomes active compound | 5.0% |
| (0.2% aglycones) | |
| PEG-60 Hydrogenated Castor Oil | 1.3% |
| Preservatives, Sodium Hydroxyde Solution, Perfume, Aqua | ad 100.0% |

11. Anti-acne tincture

| | |
|---|---|
| Soya isoflavone aglycone/liposomes active compound in | 20.0% |
| alcohol (2% aglycones) | |
| PEG-60 Hydrogenated Castor Oil | 10.0% |
| Preservatives, Aqua | ad 100.0% |

12. Whitening-Creme

| | |
|---|---|
| Ethylhexyl Methoxycinnamate | 6.5% |
| Caprylic/Capric Triglyceride | 4.0% |
| Arctostaphylos Uva Ursi | 2.0% |
| Glycerin | 2.0% |
| CI 77891 | 2.0% |
| Butyl Methoxydibenzoylmethane | 1.5% |
| Dimethicone | 1.5% |
| PEG-20 Methyl Glucose Sesquistearate | 1.2% |
| Soya isoflavone aglycone/liposomes active compound | 5.0% |
| (0.2% aglycones) | |
| Methyl Glucose Sesquistearate | 1.0% |
| Nylon-12 | 0.8% |
| Cetyl Alcohol | 0.75% |
| Stearyl Alcohol | 0.75% |
| Preservatives, Perfume, Aqua | ad 100.0% |

13. After-Shave balm against signs of skin

| | |
|---|---|
| Hamamelis virginiana Extract | 3.0% |
| PEG-20 Methyl Glucose Sesquistearate | 2.7% |
| Glycerin | 2.0% |
| Aluminium Starch Octenyl Succinate | 2.0% |
| Oleyl Oleate | 2.0% |
| Glucose | 2.0% |
| Soya isoflavone aglycone/liposomes active compound | 5.0% |
| (0.2% aglycones) | |
| Methyl Glucose Sesquistearate | 0.9% |
| Mentyl Lactate | 0.4% |
| Allantoin | 0.4% |
| Preservatives, Perfume, Aqua | ad 100.0% |

What is claimed is:

1. A cosmetic comprising at least one isoflavone aglycone in a biologically active form as active component, said at least one isoflavone aglycone being incorporated into liposomes.

2. The cosmetic of claim 1, wherein said at least one isoflavone aglycone is selected form the group consisting of genistein and daidzein.

3. The cosmetic of claim 1 or 2 further comprising at least one algal extract

4. The cosmetic of claim 3, wherein said at least one algal extract is an extract of algae of the genus Spirulina.

5. The cosmetic of claim 1, wherein the concentration of said isoflavone aglycone is from 1 to 500 mg per kg of the cosmetic.

6. The cosmetics of claim 5, wherein the concentration of said isoflavone aglycone is from 20 to 100 mg per kg of the cosmetic.
